(19) United States
(12) Patent Application Publication
(10) Pub. No.: US 2026/0271969 A1
(43) Pub. Date: Sep. 17, 2026

(54) Accelerated Start Cow Treatment

(71) Applicant: Royden Anderson, Didsbury (CA)

(72) Inventor: Royden Anderson, Didsbury (CA)

(21) Appl. No.: 19/558,614

(22) Filed: Mar. 6, 2026

Related U.S. Application Data
(60) Provisional application No. 63/769,859, filed on Mar. 11, 2025.

(51) Int. Cl. A23K 20/24; A23K 20/105; A23K 20/142; A23K 20/174; A23K 20/20; A23K 20/26; A23K 50/10; A61K 31/047; A61K 31/07; A61K 31/198; A61K 31/355; A61K 31/375; A61K 31/4415; A61K 31/455; A61K 31/51; A61K 31/525; A61K 31/593; A61K 31/714; A61K 33/14; A61K 33/30; A61K 33/32; A61K 33/34; A61K 33/42

(52) U.S. Cl. CPC A23K 20/24; A23K 20/105; A23K 20/142; A23K 20/174; A23K 20/26; A23K 20/30; A23K 50/10; A61K 31/047; A61K 31/07; A61K 31/198; A61K 31/355; A61K 31/375; A61K 31/4415; A61K 31/455; A61K 31/51; A61K 31/525; A61K 31/593; A61K 31/714; A61K 33/14; A61K 33/30; A61K 33/32; A61K 33/34; A61K 33/42

(57) ABSTRACT

An aqueous nutritional composition is administered to a dairy cow following calving to accelerate recovery and milk production readiness. The composition comprises propylene glycol, calcium, phosphorus, magnesium, potassium, copper, zinc, manganese, cobalt, and a comprehensive blend of vitamins including A, D, E, and B-complex, formulated in a water-based liquid that she rapidly absorbs. The composition is administered from a bottle containing a one-pint dose (440 ml) at calving and at 12-hour intervals for two to three days. The composition restores appetite, normalizes calcium and glucose levels, and addresses multiple post-calving deficiencies such as milk fever and ketosis. The treatment method provides a faster response and recovery compared to conventional treatments, and delivers complete mineral and vitgamin supplementation in a single product.

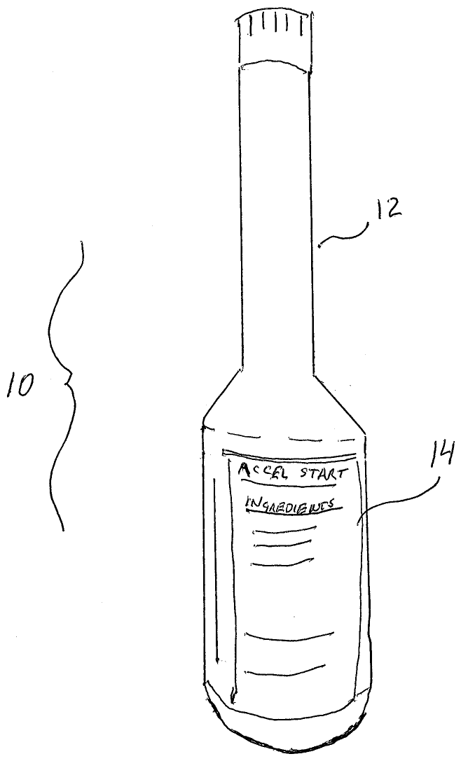

This patent application claims the benefit under 35 U.S.C. 113(e) of my/our provisional U.S. patent application, Ser. No. 63/769,859, filed Mar. 11, 2025. The disclosure in that provisional application is incorporated by reference herein.

The present invention concerns treatment of cows following calving. Products according to the present invention are different from other post calving treatments for cows, and these differences make a significant improvement in getting the cow started to a milk production condition. These can be explained in fairly simple terms.

Improvements include faster appetite and faster recoveries. The product has even been proven to work when intravenous products were not an option for recovery and the cow is given our product. The advantages are faster response time, faster recoveries and also dealing with different ailments all at once where competing products only deal with one ailment. These products, Accel Start and Full Throttle, are designed to deal with multiple deficiencies, sickness, or ailments. Thus giving the farmer the ease of mind that no matter what is wrong with the cow, he only has to pick this product to give and will cover all the mineral vitamin and sugar deficiencies whereas before he had to treat this one at a time, trying to figure out what the deficiency is. Basically, these are an all in one product doing multiple things and doing it faster than any other product on the market, even if the other product actually does multiple things.

### BRIEF DESCRIPTION OF THE DRAWING

The sole FIGURE of drawing illustrates one embodiment of a single-dose bottle from which the treatment is administered.

### DESCRIPTION OF PREFERRED EMBODIMENTS

Full Throttle and Accel Start products: Descriptions and Advantages

The standard dosage product (Accel Start):
each dose (each 440 ml bottle) contains (approximately):
Glycol—100 ml; Calcium—40 g; Phosphorus—5 to 20 g; Mg—2.5 to 5 g; K—470 mg; Cu—425 mg; Zn—547 mg; Mn—398 mg; Co—26 mg; Vit. A—120,000 IU; Vit. D—60,000 IU; Vit. E—3500 IU; Vit. B1—500 mg; Vit. B2—150 mg; Vit. B3—4 g; Vit. B6—1,000 mg; Vit. B12—10,000 mcg. High levels of niacin (vitamin B-3) inhibits the mobilization of fatty acids (ipolylsis), which effectively reduces the precursors necessary for ketone body production, thus reducing ketones.

These ingredients support fresh cow starts, and provide the cow with normal calcium levels, normal glucose levels, a normal appetite, and deal with external stressors, where the whole bottle (the bottle's full contents) of product (440 ml) is given at the time of calving and at 12 hour intervals for two to three days.

The composition can be given directly from the bottle, or mixed in water to be drenched. The product is given to cows at time of calving or when a cow is down (subclinical or clinical). The combination of B-vitamins, Zinc, and Magnesium serve as a synthetic appetite enhancer. This mechanism supports the microbial population (e.g., in the rumen) to process feed rapidly. This triggers the cow's natural hunger signal, to accelerate the return to full dry matter intake.

For dealing with milk fever, the product contains chemicals for calcium, magnesium, and phosphorous deficiencies.

The product is designed to avoid trace mineral depletion for all ailments and start up deficiencies or even just a boost. For this purpose the product includes copper, zinc, manganese, cobalt, potassium and magnesium which are present in effective amounts.

Macro minerals include calcium, phosphorous, and magnesium (sometimes called a macro) as a nutritional supplement or for deficiencies.

Propylene glycol serves for prevention of sugar related illnesses such as ketosis. Ketosis and milk fever problems are dealt with by a complete balance of minerals, vitamins and Glycol. This gives the cow the complete nutrition she needs to battle the illness instead of just calcium or glycol which seems to be the industry standard ketosis/milk-fever treatment.

Calcium is known and studied to help prevent mastitis in fresh cows. It benefits the nerve conduit to help the nerves communicate. It keeps the muscles and nerve endings from paralyzing. Calcium assists main muscles and teat end nerves and uterine tract muscles to prevent retained placentas. Both 440 ml bottles of product (Accel Start and Full Throttle) give 40 grams calcium, with the prescribed dose (twice a day) making up 80 grams a day.

Milk fevers are usually 40 percent associated with calcium deficiencies, 20 percent phosphorus deficiency, 20 percent magnesium deficiency, and 20 percent combination of two or all three.

Ketosis is a glucose deficiency and the included 300 ml of propylene glycol will get the cow to normal glucose levels for approximately 24 hrs. Thus a dosage of two bottles a day of the more concentrated product (Full Throttle) provides the cow with 340 ml. of propylene glycol.

Trace minerals during milk fevers and ketosis are also diminished and a dosage of two per day of this product will give twice what is required. The calcium in the liquid replaces the calcium bolus (current standard treatment) that is currently given in the form of a large solid pill, that can be difficult for the cow to swallow. As for antioxidant and immune support, vitamin C works synergistically with vitamin E and Zinc t neutralize reactive oxygen species (ROS). Normally, ruminants synthesize vitamin C, but the extreme oxidative stress of the transition period, and onset of lactation, requires exogenous vitamin C to meet metabolic demands. This supplementation provides physiological support, and mitigates inflammatory responses like udder edema, and provides cellular support for mammary tissues.

Other ingredients are as follows:

### B-vitamins:

B-1 Thiamine: more than three times than the industry average in one bottle;
B-2. Riboflavin: usually never given for a cow start in this industry but gives a good result for milk production boost;
B-3 Niacin: Especially helpful for treating ketosis, helping regulate sugar levels in the blood;
B-6: (usually never given in this industry) Pyrodoxine helps with inflammation and water retention, and helps prevent Edema;
B-12: gives energy and assists energy utilization;
   Vitamin C (in the stronger product—“Full Throttle”) helps with swelling and edema, mastitis, and is a good antioxidant.

Both the standard and stronger bottled product have good antioxidant levels of copper and zinc for battling infections or bacteria related illness.

The combination of zinc, B-12, niacin, magnesium, and cobalt serves as an appetite enhancer to get the cow eating faster and quicker for fast recoveries or transitions from calving.

The sole FIGURE of drawing shows a pint bottle 10 (with volume of about 440 ml) that stores a single dose of the liquid treatment. The bottle 10 has an elongted neck 12 long enough to be inserted to the cow's throat or deep into her mouth so that the treatment can be administered easily and painlessly. The body of the bottle also bears a label 14 carrying instructions, and a list of the ingredients.

In conclusion the bottles give a good fresh cow start with all nutrient requirements needed for any problem. Full Throttle is the more powerful bottle for subclinical or clinical cases. Accel Start is for normal cow start up cases and slightly subclinical.

Improved delivery advantage and bio-availability. Both bottles battle milk fevers and slight ketosis problems and both deliver fresh cow start boosts and supplemental requirements. Full Throttle is more powerful than Accel Start for those hard-to-do cases and clinical cows and edema. This product not only provides the cow with all the vitamins and minerals that the cow needs for daily nutrition, but also quickly restores her appetite, giving the cow a faster intake of feed so she is up and growing healthier and stronger after calving.

The usual dosage for a cow that has calved recently is two bottles per day for up to 3 days. After that she is to be reassessed before continuing.

The trace minerals include glycine amino acids in liquid form (the fastest absorbed and soluble amino acid) and considered best in market.

Vitamins are all water soluble for fast absorption. Dry vitamins are fully solubilized as a liquid vitamin and mineral matrix.

Propylene glycol (which is industry standard)

Calcium chloride, considered to be the fastest absorbed calcium (absorbed in blood stream within 30 minutes).

The liquid system bypasses the lag time required for dry particulates to dissolve, ensuing nutrients are immediately available during high-stress periods.

Phosphorus: a combination of phosphoric acid or an alkaline phosphorous salt, e.g. monosodium phosphate or trisodium phosphate to make up phosphate requirements. This combination of the two phosphates keeps the product from agitating the cow's throat or burning. The regular product (Accel Start)—5 grams; The stronger product (Full Throttle)—20 grams (per bottle). Phosphorus source include a combination of phosphoric acid and alkaline phosphate salts, e.g., monosodium phosphate. The contents thus are a buffered solution, which maintains a physiological pH to prevent mucosal irritation or burning. Also, the phosphorus ions to cellular energy transfer (ATP) and to muscular function.

Some prior or existing treatments include some of these ingredients but are just added to the cow's drinking water. In that case none will act as fast as the combination of ingredients of this invention, all given to the cow in liquid form. The product of this invention is designed to work within about 30 minutes of giving the bottle of the product to the cow, and for up to 24 hrs. Other (currently existing) products given mixed in water will take a lot longer for the cow to absorb.

In a practical example of this post-calving treatment, each 440 ml dose contains (approximately) glycol (170 ml; calcium—minimum concentration—5.89%); phosphorus (min.−2.84%); magnesium (min. 0.80%); copper (min. 913 ppm); zinc (min. (65 ppm) ; manganese (min. 443 ppm); cobalt (min. 43 ppm); potassium ((min. 785 ppm); vitamin A (Min. 180,000 IU/kg=81.818 IU/lb); vitamin D (min. 9,000 iU/kg=4091 IU/lb); vitamin E (min. 5345 IU/kg=2430 IU/lb); B vitamins as thiamine (B1) (min. 800 ppm); riboflavin (B2) (min. 225 ppm); niacin (B3) (min. 71670 ppm); Vit. B12 (min. 1670 mcg/kg=7590 mcg/lb).

This formulation as specified is designed as a comprehensive metabolic support tool for dairy cows during their “transition period” (the time around calving) or during bouts of illness. At this stage, a cow's body undergoes massive physiological stress, often leading to a “negative energy balance” and a sudden drop in blood calcium levels.

The breakdown of this formulation explains how it is effective for resolving milk fever, ketosis, and sluggish rumen function:
(A) Resolving Ketosis with Glycol (170 ml)—Ketosis occurs when a cow cannot eat enough to meet her energy demands, causing her to burn body fat too quickly, and thereby produces toxic ketones. Here the 170 ml of glycol (typically propylene glycol) acts as a direct glucose precursor. Once ingested, it is rapidly absorbed and converted by the cow's liver into glucose. This provides an immediate energy spike, raises blood sugar, and signals the body to stop breaking down fat, thus effectively reversing the ketosis cycle.
(B) Resolving Milk Fever, with calcium chloride (providing 40 grams elemental Ca): Milk fever (hypocalcemia) occurs when the demand for calcium for colostrum and milk production exceeds the cow's ability to mobilize it from her bones. Calcium chloride is optimum for this as oral treatment as calcium chloride is highly soluble, thus creating a rapid spike in the blood calcium levels within 30 to 60 minutes after administration. The 40 gram dose is a potent loading dose that helps bridge the gap until the cow's own hormonal system can begin to mobilize calcium from her skeleton.
(C) Boosting rumen function and appetite is a role for the b vitamins and cobalt. A sick cow or a fresh cow often fails to eat anything, and this can cause the rumen to slow down(i.e., stasis) . Thus this formulation re-starts the eating and rumen cycle.
(C1) Niacin (B3) at high levels, reduces ketone production and helps the liver process fats more efficiently. (C2) Cobalt is essential for the rumen microbes to synthesize their own Vitamin B 12, without which (the cobalt) the rumen would slow down. (C3) Thiamine (B1) and Riboflavin (B2) are critical for carbohydrate metabolism. These vitamins help the rumen microbes break down fiber more efficiently. Thus, supporting the microbial population allows the rumen to process the cow's feed faster. As the rumen empties more quickly, though efficient fermentation, the cow's natural hunger signal is triggered, bringing her back on feed sooner.
(C4) Breakdown of the supporting ingredients: Phosphorus (2.84%)—Often, cows with low calcium will also have low phosphorus, i.e., hypophosphatemia. Providing phosphorus helps with muscle function and energy transfer at the cellular level. Magnesium (0.80%): Magnesium is a necessary co-factor for calcium mobilization. A cow cannot mobilize her own bone calcium if her magnesium levels are low. The magnesium ingredient ensures the calcium in the 440 ml dose is effective, and thus, in her body, the calcium is actually useable. Magnesium is essential for the secretion of parathyroid hormone (PTH), and to the sensitivity of PTH receptors to draw calcium from bone reserves. Potassium is an essential electrolyte that is often lost during calving or through diarrhea when a cow is sick. The included potassium helps maintain fluid balance and proper nerve function.
Vitamins A, D, and E:
Vitamin D is critical for the long-term absorption of calcium from the gut.
Vitamins E and A are powerful antioxidants that support the immune system, which is naturally suppressed during calving, so this ingredient helps to prevent secondary infections, such as mastitis.

Trace minerals (Zinc, Copper and Manganese) are co-factors for hundreds of enzymes in the cow's body that manage issues from tissue repair after calving to immune defense. Selenium may assist or boost the effect of Vitamin E.

In summary, the product's calcium chloride provides immediate nerve/muscle function (e.g., addressing milk fever); Glycol provides immediate energy (addressing ketosis); and a heavy dose of B-vitamins and cobalt stimulates the rumen. Thus this formulation addresses the three biggest hurdles a fresh cow faces. Administration of this formulation shifts her from a state of metabolic “shutdown” back into a state of production.

What is claimed is:

1. An orally administered aqueous nutritional composition in a volume of 440 ml to one pint, for post-calving treatment of a dairy cow, comprising:
effective amounts of propylene glycol; calcium; phosphorus; magnesium; potassium; copper, zinc, manganese, and cobalt, wherein the aqueous solution is substantially one pint in volume; and further comprising at least one of vitamins of the group consisting of Vitamin A; Vitamin D; vitamin E, Vitamin B1; Vitamin B2, Vitamin B3, Vitamin B6, and Vitamin B12,
Such that said aqueous nutritional composition is adapted for rapid absorption of the components thereof and is effective to restore appetite, normalize calcium and glucose levels, and reduce post-calving deficiencies in the cow.

2. The aqueous nutritional composition according to claim 1 wherein said effective amounts are present as
about 100 ML to 200 ML of propylene glycol;
about 40 g of calcium;
about 5 g to 20 g of phosphorus;
about 2.5 g to 5 g of magnesium;
about 470 mg of potassium;
about 425 mg of copper;
about 547 mg of zinc;
about 398 mg of manganese;
about 26 mg of cobalt; and
said vitamins are present as
Vitamin A in an amount of about 120,000 IU,
Vitamin D in an amount of about 60,000 IU,
Vitamin E in an amount of about 3500 IU,
Vitamin B1 in an amount of about 500 mg,
Vitamin B2 in an amount of about 150 mg,
Vitamin B3 in an amount of about 4 g,
Vitamin B6 in an amount of about 1,000 mg, and
Vitamin B12 in an amount of about 10,000 mcg; respectively.

3. The aqueous nutritional composition according to claim 1, wherein the calcium is provided as calcium chloride, effective for rapid absorption by the cow within about 30 minutes.

4. The aqueous nutritional composition according to claim 1, wherein the phosphorus is present as a combination of phosphoric acid and an alkaline phosphate salt selected from monosodium phosphate and trisodium phosphate.

5. The aqueous nutritional composition according to claim 1, further comprising glycine amino acids in liquid form and adapted to enhance mineral absorption.

6. The aqueous nutritional composition according to claim 1, wherein the vitamins are water-soluble sufficiently to achieve absorption rates greater than 80%.

7. The aqueous nutritional composition according to claim 1, wherein the composition additionally includes Vitamin C in an amount effective to reduce edema and provide antioxidant benefits.

8. The aqueous nutritional composition according to claim 1 further comprising a bottle containing one dose of said composition and said bottle having a capacity of one pint, and with an elongated neck to be placed into the mouth of the cow.

9. A method of treating a cow following calving, comprising:
Orally administering to the cow an aqueous nutritional composition in a volume of substantially 440 ml to one pint, comprising propylene glycol, calcium, phosphorus, magnesium, potassium, copper, zinc, manganese, cobalt, and vitamins selected from the group consisting of Vitamins A, D, E, B1, B2, B3, B6, and B12;
wherein the composition is administered in a dosage of about 440 ML at the time of calving and repeated at intervals of about 12 hours for a period of two to three days;
thereby restoring appetite, normalizing calcium and glucose levels, and reducing post-calving deficiencies including milk fever and ketosis.

10. The method of claim 9, wherein the composition contained in a bottle with an openable neck and is administered directly from the neck of the bottle into the mouth of the cow.

11. The method of claim 9 wherein said composition is mixed with water and provided to the cow by drenching.

12. The method of claim 9, comprising administering the aqueous nutritional composition at half-day intervals, such that the cow receives two doses per day for up to three days, followed by reassessment of her post-calving health.

13. The method of claim 9, comprising preventing retention of placenta by maintaining uterine muscle function through calcium supplementation.

14. The method of claim 9, comprising enhancing the treated cow's appetite by supplying her a combination of zinc, niacin, magnesium, cobalt, and Vitamin B12.

15. The method of claim 9 comprising administering the aqueous nutritional composition such that the treatment is configured to bat effective in restoring the cow's health and appetite within about 30 minutes of administration such that she maintains normal glucose levels for up to 24 hours.
